(19) United States
(12) Patent Application Publication
(10) Pub. No.: US 2026/0237498 A1
(43) Pub. Date: Aug. 13, 2026

(54) EQUITY PLANNING FOR HEALTHCARE PROVIDER

(71) Applicant: BLUE CROSS AND BLUE SHIELD OF MASSACHUSETTS, INC., Boston, MA (US)

(72) Inventor: Mark Friedberg, Boston, MA (US)

(21) Appl. No.: 19/470,970

(22) PCT Filed: Apr. 1, 2024
(86) PCT No.: PCT/US2024/022533

§ 371 Date: Sep. 30, 2025

Related U.S. Application Data
(60) Provisional application No. 63/493,617, filed on Mar. 31, 2023.

(51) Int. Cl. G16H 40/20; G06Q 10/0639

(52) U.S. Cl. CPC G16H 40/20; G06Q 10/0639

(57) ABSTRACT

A method of assigning a score to a provider reads a first size and a first performance score associated with each group in a baseline time period, and a second size and a second performance score associated with each group in a measurement time period. The method filters for groups whose first and second size exceeds a predetermined size threshold and whose first and second performance score exceeds a predetermined performance threshold. The method selects a reference group. A first average inequity is determined for each of the plurality of groups relative to the reference group based on their respective first size and first performance score. A second average inequity is determined for each group relative to the reference group based on their respective second size and second performance score. An EIM score is determined based on a percentage change in inequity between the first and the second average inequities.

### CROSS-REFERENCE TO RELATED APPLICATIONS

This application is a national stage entry under 35 U.S.C. § 371 of International Application No. PCT/US24/22533, filed on Apr. 1, 2024, and claims the benefit of priority to U.S. Provisional Application. No. 63/493,617, filed Mar. 31, 2023, which is hereby incorporated by reference in its entirety.

### BACKGROUND

In the health care industry, provider organizations may be parties to contracts that provide financial incentives for improving of delivered services. Such contracts may include terms having financial incentives linked to provider organization performance on measures of quality and total costs of care.

### BRIEF SUMMARY

According to embodiments of the present disclosure, methods of and computer program products for determining an equity improvement measure (EIM) are provided.

In some embodiments, a method of assigning an Equity Incentive Measure (EIM) score to a provider can include reading a first size and a first performance score associated with each of a plurality of groups in a baseline time period. The method can include reading a second size and a second performance score associated with each of the plurality of groups in a measurement time period. The method can include filtering the plurality of groups to those whose first and second size exceeds a predetermined size threshold. The method can include filtering the plurality of groups to those whose first and second performance score exceeds a predetermined performance threshold. The method can include selecting one of the plurality of groups as a reference group. The method can include determining a first average inequity for each of the plurality of groups relative to the reference group based on their respective first size and first performance score. The method can include determining a second average inequity for each of the plurality of groups relative to the reference group based on their respective second size and second performance score. The method can include determining an EIM score based on a percentage change in inequity between the first and the second average inequities.

In some embodiments, determining the first average inequity for each of the plurality of groups can include (1) determining an equity weight for each of the plurality of groups relative to the reference group based on their respective first size and first performance score, (2) determining a weight for each of the plurality of groups relative to the reference group, based on their respective first size, and (3) determining a baseline inequity for each of the plurality of groups relative to the reference group based on their respective first size and first performance score.

In some embodiments, determining the second average inequity for each of the plurality of groups can further include (1) determining an equity weight for each of the plurality of groups relative to the reference group based on their respective second size and second performance score, (2) determining a weight for each of the plurality of groups relative to the reference group, based on their respective second size, and (3) determining a baseline inequity for each of the plurality of groups relative to the reference group based on their respective second size and second performance score.

In some embodiments, determining the EIM score is a constant if the percentage change in inequity is at or above a threshold. In some embodiments, the EIM score is a product of the percentage change in inequality between the first and second average inequities and a linear constant if the percentage change in inequity is below the threshold.

In some embodiments, the first performance score and second performance score are each a quality improvement metric.

In some embodiments, the method further includes reading a contract term of a contract with the provider. In some embodiments, the method further includes determining a compliance threshold in the contract term. In some embodiments, the method further includes comparing the EIM score to the compliance threshold. In some embodiments, the method further includes providing an indication of compliance or non-compliance with the contract based on the comparing.

A non-transitory computer readable storage medium storing executable instructions that, when executed by one or more processors, cause the one or more processors to perform any of the above methods.

In some embodiments, a system includes a computing node comprising a computer readable storage medium having program instructions embodied therewith. In some embodiments, the program instructions are executable by a processor of the computing node to cause the processor to perform any of the above methods. In some embodiments, the computing node is a contract compliance server.

### BRIEF DESCRIPTION OF THE SEVERAL VIEWS OF THE DRAWINGS

FIG. 1 is a flowchart illustrating a method of calculating an EIM score according to embodiments of the present disclosure.

FIG. 2 is a flowchart illustrating an example of a method of running a simulation to determine whether a condition is satisfied for a provider group according to embodiments of the present disclosure.

FIG. 3 is a graph of the EIM score plotted against the PRW in accordance with embodiments of the present disclosure.

FIG. 4 is a graph illustrating PRW calculating using imputed data plotted against PRW plotted using self-reported data.

FIG. 5 is a flowchart illustrating a method of using the EIM score to comply with a contract according to embodiments of the present disclosure.

FIG. 6 is a computing node according to embodiments of the present disclosure.

### DETAILED DESCRIPTION

In some cases, health care contracts (e.g., between a provider and an insurance company, or a provider and a governmental agency) may include financial incentives linked to measures of racial and ethnic equity of care. The contractual terms of these financial incentives linked to the measures of racial and ethnic equity of care can be similar to the contractual terms linked to measures of overall quality of care. In some embodiments, these contractual terms use a percentage of a quality-equity score (e.g., as a stand-alone performance incentive and as a factor that affects the risk share for the cost component of the contract). In some embodiments, a method of determining such a quality-equity score are described below. In some embodiments, a method of complying with contracts based on the quality-equity score is also described below.

In some embodiments, an Equity Incentive Measure (EIM) is provided as a metric to be used in such contractual terms. In some embodiments, a method can calculate the Equity Incentive Measure (EIM) Score. In some embodiments, a method can determine determining a Measure's Eligibility for the EIM Score. In some embodiments, a method can determine a function or curve shape for the EIM Score.

In some embodiments, the below methods are provided to calculate an EIM score. In some embodiments, however, as the contractual terms may change, the method of calculating the EIM score may vary.

In some embodiments, the EIMs described herein are based on data of quality measures that are stratified for race, ethnicity, or both. In some embodiments, every EIM has a corresponding overall quality measure incentive. For example, in a given contract there can be an EIM that incentivizes reduction of racial and ethnic inequities in blood pressure control, then the given contract can include an incentive to improve overall performance (e.g., based on aggregated data for members of all races and ethnicities) on the same measure of blood pressure control.

In some embodiments, while the EIM calculations described below describe measures stratified on racial and ethnic inequities, the methods described herein can be applied to future changes in how races and ethnicities are categorized, to other bases of inequities (e.g., language, sexual orientation, gender identity, national origin, disability status), and to intersections between current and future bases of inequities. In some embodiments, any basis for stratifying quality measure performance data can be applied to an EIM score for a goal of reducing inequality.

### Calculating an Equity Incentive Measure (EIM) Score

### Section I. Data Elements

In some embodiments, quality incentive measures of a practice organization can serve as initial data for calculating an Equity Incentive Measure (EIM) score. A quality incentive measure can measure a healthcare process, a healthcare outcome, patient-reported data, or organizational data that is associated with the ability to provide high-quality health care. In some embodiments, these quality incentive measures can be stratified for racial or ethnic patients or providers. In some embodiments, the quality incentive measures (e.g., measurements, data points) can be data used to calculate the EIM score.

In some embodiments, various data elements (e.g., the quality incentive measures) are used to calculate an Equity Incentive Measure (EIM) score. These various data elements can include an index i for a measurement year. The measurement year can either be baseline (“base”) or applicable quality measurement period (“MP”). The latter represents a measurement period any of the years for which performance is measured under a contract. In some embodiments, the “denominator” refers to a number of patients, providers, staff, combination of the above, etc. that meet the stratification criteria (e.g., a member of the racial or ethnic stratum).

For example, a baseline measurement yar may be the calendar year 2023, but a MP may be Jun. 1, 2022-May 31, 2023 as defined by a contract or other agreement that requires measurement of equity measures. In some embodiments, the EIM uses n_{G,i} as the denominator vale for members classified to a racial or ethnic group G for measurement period i. In some embodiments, the EIM uses p_{W,i} as the stratified performance value for members classified to the racial or ethnic group G for measurement period i.

In some embodiments, for four racial/ethnic groups W, X, Y, Z, the Equity Incentive Measure (EIM) score calculation can be based on the following data elements collected at baseline year and/or at applicable quality measurement period (Measurement Period; there is one Measurement Period for each year of the contract) for the EIM in question:
n_{W,i} is the i denominator value for members classified to racial/ethnic group W
p_{W,i} is the i stratified performance for members classified to racial/ethnic group W
n_{X,i} is the i denominator value for members classified to racial/ethnic group X
p_{X,i} is the i stratified performance for members classified to racial/ethnic group X
n_{Y,i} is the i denominator value for members classified to racial/ethnic group Y
p_{Y,i} is the i stratified performance for members classified to racial/ethnic group Y
n_{Z,i} is the i denominator value for members classified to racial/ethnic group Z
p_{Z,i} is the i stratified performance for members classified to racial/ethnic group Z

In some embodiments, the data to compute the EIM score is shown by Table 1, below.

TABLE 1
Data used to generate EIM Score

| Racial or | Denominator | | Stratified Performance | |
|---|---|---|---|---|
| Ethnic | | Measurement | | Measurement |
| Group | Baseline | Period | Baseline | Period |
| W | n_{W, base} | n_{W, MP} | p_{W, base} | p_{W, MP} |
| X | n_{X, base} | n_{X, MP} | p_{X, base} | p_{X, MP} |
| Y | n_{Y, base} | n_{Y, MP} | p_{Y, base} | p_{Y, MP} |
| Z | n_{Z, base} | n_{Z, MP} | p_{Z, base} | p_{Z, MP} |

### Section II. Calculating EIM

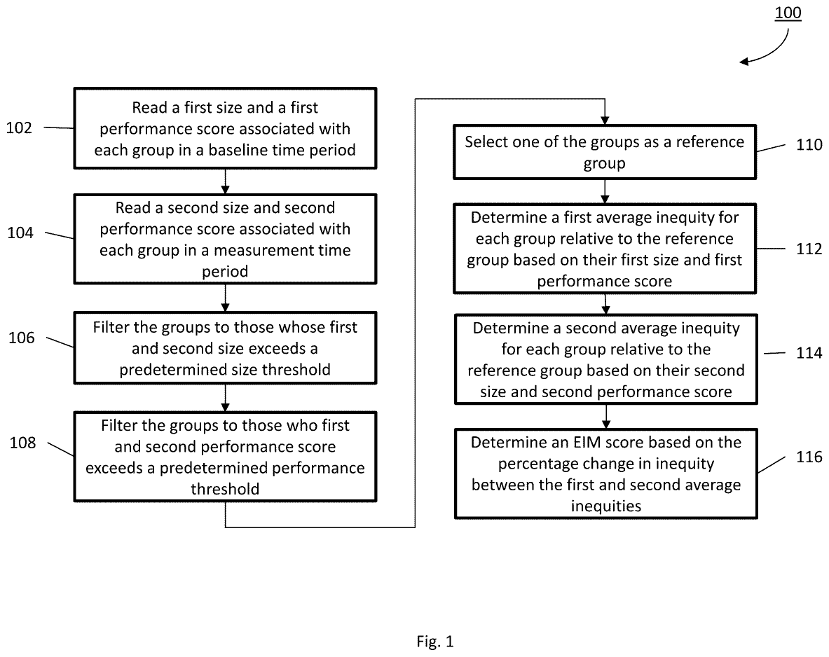

FIG. 1 is a flowchart 100 illustrating a method of calculating an EIM score according to embodiments of the present disclosure. In some embodiments, the method can be performed by a server or other computer. In some embodiments, calculations are performed in the baseline time period using data from that baseline time period. The server can read a first size and a first performance score associated with a first baseline period 102.

The server can read a second size and a second performance score associated with each group in a measurement time period 104. The server can filter the groups to those whose first and second size exceeds a predetermined size threshold 106. In some embodiments, racial/ethnic groups are selected to be included as part of the EIM score calculation by identifying racial/ethnic strata having baseline denominator sizes greater than or equal to a threshold value (e.g., identify which of n_{W,base}, n_{X,base}, n_{Y,base}, n_{Z,base} are ≥90). In some embodiments, the threshold is 90. In some embodiments, values other than 90 can be employed as the threshold. Any racial/ethnic strata having baseline denominators less than the threshold can be removed. In some embodiments, if only one racial/ethnic stratum has a baseline denominator greater than or equal to 90, then the entire measure is not eligible for an EIM score.

The server can filter the groups to those who first and second performance score exceeds a predetermined performance threshold 108. The server can select one of the groups as a reference group 110. In some embodiments, the reference group for an EIM is a racial/ethnic stratum with a largest number of members (e.g., largest baseline denominator). In some embodiments, by having the reference group be a group with the largest, at least one of the rates used to calculate the EIM is based on a relatively large value.

The server can determine a first average inequity for each group relative to the reference group based on their first size and first performance score 112. The server can determine a second average inequity for each group relative to the reference group based on their second size and second performance score 114. The server can then determine the EIM score based on the percentage change in inequity between the first and second average inequities 116.

In some embodiments, Group Z of Table 1 can be the reference group and all four racial/ethnic groups (e.g., W, X, Y, Z) meet the threshold to be included in the EIM score calculation for this measure. In some embodiments, the following definitions can be used in this scenario, or in cases where two, three, or greater than four racial/ethnic groups are included in the EIM score calculation.

### Definitions

Minimum Denominator Requirement (MDR): In some embodiments, a MDR is calculated at 70%-100% of denominators in a baseline year (e.g., MY2019) based on simulations examining the degree of variability in the measure. Some measures have large enough baseline denominators and/or large enough baseline inequities to be able to lower their denominator sizes without major changes to the variability of the measure. In some embodiments, the MDR being 70, 80, 90, or 100% provides selecting measures that have large enough baseline denominators. In some embodiments, r_{MDR} is a constant where r_{MDR}∈{0.7, 0.8, 0.9, 1.0} and the MDR is equal to r_{MDR}(n_{t,base}) for each t∈{W, X, Y, Z}.

Minimum Performance Required (MPR): In some embodiments, MPR is a minimum accepted value for the Measurement Period Stratified Performance for each race/ethnicity category. The MPR is incorporated to ensure that rewards are not given for relative equity improvements that are a result of decreasing performance for some racial/ethnic groups (e.g., if White performance is higher than Hispanic performance, Hispanic-White inequity could theoretically be improved by decreasing performance for White members). In some embodiments, a Bonferroni-corrected two-sided

$$( 1 - \frac{α}{{num}_{RE}} )$$

% Wald confidence intervals is calculated using n_{t,base}, p_{t,base} for each t∈{W, X, Y, Z}. In some embodiments, with α=0.05, num_{RE} is equal to the number of racial/ethnic strata that remains after applying the minimum baseline denominator size. In some embodiments, assuming greater values of p indicate better levels of performance, a lower bound of this confidence interval for each t∈{W, X, Y, Z} is equivalent to the MPR for the corresponding race/ethnicity. If lesser values of p indicate better levels of performance (e.g., as in “lower-is-better” measures), then the upper bound of this confidence interval is employed, and the MPR can function as a maximum.

Baseline Category Inequity: In some embodiments, an absolute value of Baseline Stratified Performance differences can be defined between each racial/ethnic group and the reference group. The Baseline Category Inequities can be, in some embodiments such as in the example groups illustrated in Table 1, as follows: abs(p_{W,base}−p_{Z,base}), abs(p_{X,base}−p_{Z,base}), abs(p_{Y,base}−p_{Z,base}).

Baseline Denominator Weights: In some embodiments, Baseline Denominator Weights can be calculated by dividing the Baseline Denominators for all groups except the reference group by the sum of the Baseline Denominators for all groups except the reference group. Baseline Denominator Weights can be normalized (e.g., take values between 0 and 1). In some embodiments, in some embodiments such as the example of Table 1, the Baseline Denominator Weights can be calculated as follows:

$$Baseline Denominator Weights = \{ \frac{{n}_{W , base}}{{n}_{W , base} + {n}_{X , base} + {n}_{Y , base}} \frac{{n}_{X , base}}{{n}_{W , base} + {n}_{X , base} + {n}_{Y , base}} , \frac{{n}_{Y , base}}{{n}_{W , base} + {n}_{X , base} + {n}_{Y , base}} \} = \{ {d}_{W} , {d}_{X} , {d}_{Y} \}$$

Baseline Category Inequity Weights: In some embodiments, Baseline Category Inequity Weights are values obtained by dividing each of the Baseline Category Inequities by the sum of the Baseline Category Inequities. Baseline Category Inequity Weights can be normalized (e.g., take values between 0 and 1). Baseline Category Inequity Weights can be calculated as follows:

$$Baseline Category Inequity Weights = \{ \frac{abs ( {p}_{W , base} - {p}_{Z , base} )}{b} , \frac{abs ( {p}_{X , base} - {p}_{Z , base} )}{b} , \frac{abs ( {p}_{Y , base} - {p}_{Z , base} )}{b} \} , = \{ {b}_{W - Z} , {b}_{X - Z} , {b}_{Y - Z} \}$$

where b=abs(p_{W,base}−p_{Z,base})+abs(p_{X,base}−p_{z,base})+abs(p_{Y,base}−p_{Z,base})

Equity Weights: In some embodiments, equity weights are applied to each racial/ethnic category when calculating a Baseline Weighted Average Inequity and a Weighted Average Inequity. In some embodiments, for a given measure, an Equity Weight for a racial/ethnic strata is a weighted (e.g., equally weighted) average of the Baseline Denominator Weights and the Baseline Category Inequity Weights for that strata. In some embodiments, this places a greater weight on racial/ethnic inequities that are larger in magnitude and/or impact a relatively larger number of members (e.g., groups having larger baseline denominators). In some embodiments, an equity weight can be calculated as follows:

$$Equity Weights = \{ \frac{{d}_{W} + {b}_{W - Z}}{2} , \frac{{d}_{X} + {b}_{X - Z}}{2} , \frac{{d}_{Y} + {b}_{Y - Z}}{2} \} = \{ {w}_{W - Z} , {w}_{X - Z} , {w}_{Y - Z} \}$$

Baseline Weighted Average Inequity: In some embodiments, a Baseline Weighted Average Inequity is calculated by multiplying the Equity Weights by the Baseline Category Inequities. Baseline Weighted Average Inequity can be calculated as follows:

$$Baseline Weighted Average Inequity = {w}_{W - Z} abs ( {p}_{W , base} - {p}_{Z , base} ) + {w}_{X - Z} abs ( {p}_{X , base} - {p}_{Z , base} ) + {w}_{Y - Z} abs ( {p}_{Y , base} - {P}_{Z , base} )$$

Calculations Completed after the Measurement Period Ends

Category Inequity: In some embodiments, a category inequity value is an absolute value of Measurement Period Stratified Performance differences between each racial/ethnic group and the reference group. The Category Inequities can be calculated as follows:

$$abs ( {p}_{W , MP} - {p}_{Z , MP} ) , abs ( {p}_{X , MP} - {p}_{Z , MP} ) , abs ( {p}_{Y , MP} - {p}_{Z , MP} ) .$$

Weighted Average Inequity: In some embodiments, a Weighted Average Inequity is calculated by multiplying the Equity Weights by the Category Inequity for each racial/ethnic group. The Weighted Average Inequity can be calculated as follows:

$$Weighted Average Inequity = {w}_{W - Z} abs ( {p}_{W , MP} - {p}_{Z , MP} ) + {w}_{X - Z} abs ( {p}_{X , MP} - {p}_{Z , MP} ) + {w}_{Y - Z} abs ( {p}_{Y , MP} - {p}_{Z , MP} )$$

### Section III. Calculating the EIM Score for a Measure Identified as Eligible for an EIM Score

In some embodiments, calculating an EIM score can be performed on any measure (e.g., a quality incentive measure) eligible to receive one. In some embodiments, an EIM score is calculated on eligible Ambulatory Care Quality Incentive Measures (AIMs).

In some embodiments, to calculate an EIM score, the baseline calculations above should be calculated for all eligible racial/ethnic strata described above (e.g., Minimum Denominator Requirement (MDR), Minimum Performance Required (MPR), Baseline Category Inequity, Baseline Denominator Weights, Baseline Category Inequity Weights, Equity Weights, and/or Baseline Weighted Average Inequity).

In some embodiments, a particular measure can be identified as eligible to receive an EIM score. Group Z can be the reference group for the particular measure. All four racial/ethnic groups of Table 1 can be included in the EIM score calculation. Once measurement period data is available, a method of calculating the EIM score calculation can include determining whether the Measurement Period denominators (n_{W,MP}, n_{X,MP}, N_{Y,MP}, n_{Z,MP}) are each greater than or equal to the corresponding MDR. In some embodiments, if the Measurement Period denominators are not greater than or equal to the MDR, then this measure is no longer eligible to receive an EIM score, and no further calculation is performed. In some embodiments, with such a condition, the measure is no longer eligible for two potential reasons. First, large drops in the measurement period denominators may indicate that the variability in measuring improvements in inequities is too large. Second, significant drops in measurement period denominators for particular racial/ethnic categories may indicate that the practice group is intentionally dropping certain members to perform better on the measure. In some embodiments, therefore with the Measurement Period denominators being less than the MDR, the EIM is excluded from the Aggregated Weighted EIM Score.

In some embodiments, the Measurement Period denominator sizes are greater than or equal to their corresponding MDRs to proceed with the calculation of this specific EIM score. It can be recognized that the Measurement Period denominator sizes are greater than or equal to their corresponding MDRs for the remainder of this example.

In some embodiments, the method determines whether the Measurement Period Stratified Performance, (p_{W,MP}, p_{X,MP}, p_{Y,MP}, p_{Z,MP}), falls below the corresponding MPR for each of the racial/ethnic strata that remain. Assuming larger values of Stratified Performance indicate higher levels of performance, a Measurement Period Stratified Performance value falling below the corresponding MPR can represents a statistically significant performance decline relative to baseline year. For a given EIM, if Measurement Period Stratified Performance p_{t,MP} for any stratum is less than its corresponding MPR, the entire measure receives an EIM score of 0 and no further calculation is performed.

In some embodiments, the Measurement Period Stratified Performance values exceed their MPRs and the method can proceed to calculate the EIM Score. The method can calculate the category inequity for each racial/ethnic group using, in this example, the reference group Z. Table 2, below, illustrates the category inequity calculations and equity rates for each non-reference group, W, X, and Y.

In some embodiments, the method can calculate the Weighted Average Inequity by multiplying the Category Inequity by the Equity Weights. Table 2 illustrates the resulting formula for the Weighted Average Inequity.

TABLE 2
EIM Calculations

| | Denominator | | Stratified Performance | | | | Category Inequity | | |
|---|---|---|---|---|---|---|---|---|---|
| | | Measurement | | Measurement | | | | Measurement | Equity |
| | Baseline | Period | Baseline | Period | MDR | MPR | Baseline | Period | Weights |
| W | n_{W, base} | n_{W, MP} | p_{W, base} | p_{W, MP} | r_{MDR} n_{W, base} | Precalculated | abs(p_{W, base} − | abs(p_{W, MP} − | w_{W-} |
| | | | | | | using n_{t, base}, | p_{Z, base}) | p_{Z, MP}) | |
| X | n_{X, base} | n_{X, MP} | p_{X, base} | p_{X, MP} | r_{MDR} n_{X, base} | p_{t, base} for t ∈ | abs(p_{X, base} − | abs(p_{X, MP} − | w_{X-} |
| | | | | | | {W, X, Y, Z} | p_{Z, base}) | p_{Z, MP}) | |
| Y | n_{Y, base} | n_{Y, MP} | p_{Y, base} | p_{Y, MP} | r_{MDR} n_{Y, base} | | abs(p_{Y, base} − | abs(p_{Y, MP} − | w_{Y-} |
| | | | | | | | p_{Z, base}) | p_{Z MP}) | |
| | n_{Z, base} | n_{Z, MP} | p_{Z, base} | p_{Z, MP} | r_{MDR} n_{Z, base} | | NA | NA | NA |

| Baseline Weighted Average Inequity | w_{W-Z}abs(p_{W, base} − p_{Z, base}) + w_{X-Z}abs(p_{X, base} − p_{Z, base}) + |
|---|---|
| | w_{Y-Z}abs(p_{Y, base} − p_{Z, base}) |
| Weighted Average Inequity | w_{W-Z}abs(p_{W, MP} − p_{Z, MP}) + w_{X-Z}abs(p_{X, MP} − p_{Z, MP}) + |
| | w_{Y-Z}abs(p_{Y, MP} − p_{Z, MP}) |

In some embodiments, the method can calculate a percent reduction in weighted baseline inequity (PRW) using the Baseline Weighted Average Inequity and Weighted Average Inequity as follows:

$$PRW = \frac{Baseline Weighted Average Inequity - Weighted Average Inequity}{Baseline Weighted Average Inequity}$$

In some embodiments, the EIM score is calculated as follows:

$$EIM Score = 6.667 \times PRW when PRW < 0.7 ; and \\ EIM Score = 5 when PRW \geq 0.75 .$$

In some embodiments, if the EIM score is less than 0, it can be set to 0, and if the EIM score is greater than 5, it can be set to 5. In other words, the EIM score can have a lower and upper bound, in some embodiments. In some embodiments, the above formula is for the EIM score using imputed data. In some embodiments, such as when self-reported race/ethnicity are available for substantial and similar proportions of members in the Baseline and Measurement Periods, a different EIM score formula may be applied.

In some embodiments, example baseline data is listed in Table 3, below.

TABLE 3
Example Baseline Data

| | Denominator | | Stratified Performance | |
|---|---|---|---|---|
| | | Measurement | | Measurement |
| | Baseline | Period | Baseline | Period |
| Asian | 300 | | 0.787 | |
| Black | 600 | | 0.697 | |
| Hispanic | 500 | | 0.623 | |
| White | 1800 | | 0.728 | |

In some embodiments, based on the baseline data of Table 3, the reference group is White for this example because it has the largest baseline denominator. In some embodiments, all four racial/ethnic groups are maintained in the calculation because the baseline denominator values are each greater than 90. In some embodiments, this measure has been identified as eligible for an EIM score, following the simulation procedure detailed in further detail below, and that r_{MDR}=0.8.

In some embodiments, based on the example of Table 3, the MDRs, the MPRs, the Baseline Category Inequities, the Equity Weights, and the Baseline Weighted Average Inequity can be calculated, and are illustrated in Table 4.

TABLE 4
Example Baseline Data

| | Denominator | | Stratified Performance | | | | Category | Equity Weights | |
|---|---|---|---|---|---|---|---|---|---|
| | | Measurement | | Measurement | | | Inequity | Measurement | |
| | Baseline | Period | Baseline | Period | MDR | MPR | Baseline | Period | |
| Asian | 300 | | 0.787 | | 240 | 0.728 | 0.059 | | 0.258 |
| Black | 600 | | 0.697 | | 480 | 0.650 | 0.031 | | 0.294 |
| Hispanic | 500 | | 0.623 | | 400 | 0.569 | 0.105 | | 0.448 |
| White | 1800 | | 0.728 | | 1440 | 0.702 | NA | | NA |

| Baseline Weighted Average Inequity | 0.071 |
|---|---|
| Weighted Average Inequity | |

In some embodiments, once the Measurement Period data (Denominators, Stratified Performance) becomes available, the remaining calculations can be performed to determine the weighted average inequity.

TABLE 5
Example Measurement Period Calculations

| | Denominator | | Stratified Performance | | | | Category Inequity | | |
|---|---|---|---|---|---|---|---|---|---|
| | | Measurement | | Measurement | | | | Measurement | Equity |
| | Baseline | Period | Baseline | Period | MDR | MPR | Baseline | Period | Weights |
| Asian | 300 | 308 | 0.787 | 0.784 | 240 | 0.728 | 0.059 | 0.054 | 0.258 |
| Black | 600 | 620 | 0.697 | 0.710 | 480 | 0.650 | 0.031 | 0.020 | 0.294 |
| Hispanic | 500 | 520 | 0.623 | 0.680 | 400 | 0.569 | 0.105 | 0.050 | 0.448 |
| White | 1800 | 1810 | 0.728 | 0.730 | 1440 | 0.702 | NA | NA | NA |

| | |
|---|---|
| Baseline Weighted Average Inequity | 0.071 |
| Weighted Average Inequity | 0.042 |

In some embodiments, after analyzing the data of Table 5, the measurements are eligible to receive an EIM score because the Measurement Period Denominators are each greater than or equal to their corresponding MDRs and because none of the Measurement Period Stratified Performance values are less than the corresponding MPRs.

As can be observed in the example of Table 5, the Category Inequity values are 0.054, 0.020, 0.050 and the Weighted Average Inequity is 0.042. In some embodiments, the PRW can be calculated by (0.071−0.042)/0.071=0.408. In some embodiments, the EIM score can be calculated as 0.408*6.667=2.7 since 0.408<0.75.

### Determining a Measure's Eligibility for the EIM Score

In some embodiments, criteria are established to determine which subset of the Ambulatory Care Quality Incentive Measures (AIMs) are also eligible for an EIM score for each provider group. In some embodiments, this step occurs before contracting a provider group.

In some embodiments, not all AIMs are eligible for an EIM score because of sample size or baseline inequity considerations. Together, these factors can determine the magnitude of random error in—and therefore the reliability of—each EIM measurement. In alternative methods, an AIM score for a provider group is based on between-provider comparisons among all its members eligible for a measure. In contrast, in some embodiments, the EIM score stratifies all eligible members for a measure by race/ethnicity and examines improvements in relative stratified performance rates between baseline and the measurement period. In some embodiments, the EIM score is a comparison of rates that are based on considerably smaller denominator counts than the AIM score. The smaller denominator counts can lead to increased variability in the EIM score relative to the AIM score for a specific provider. Therefore, in some embodiments, standards are used to determining which AIM measures satisfy certain minimum denominator counts and maximum estimation errors. In some embodiments, AIM measures that meet these standards are accepted to be eligible for inclusion in the set of EIMs for each provider group. In some embodiments, the approach used to apply these standards, described in further detail below, ensures reliability for each AIM.

### Section I. Overview

In some embodiments, a set of AIMs eligible for an EIM score for each provider group's contract is selected. In some embodiments, the set of AIMs eligible for an EIM score can be all of the AIMs, some of the AIMs, or none of the AIMs. In some embodiments, the set of AIMs eligible for the EIM score is based on measures that have enough members in each racial/ethnic subgroup to draw meaningful conclusions about the provider group's performance for each racial/ethnic subgroup (e.g., Condition 1) and similar expected levels of error in calculating the EIM score as what is currently accepted in calculating the AIM score (e.g., Condition 2).

In some embodiments, a simulation is conducted for each provider group that considers a range of potential improvement scenarios given the baseline data for each measure, estimates the corresponding EIM score, and then determines the distance between the estimated EIM score and the known, true value of the EIM score. In some embodiments, the simulation provides understanding of the expected levels of error in calculating the EIM score. In some embodiments, if the estimated EIM scores are close to their true value, then the error in calculating the EIM score for this measure is low. In some embodiments, the level of error for the EIM can be determined by comparing this to the level of error accepted in calculating the AIM scores at their minimum denominators. In some embodiments, if the level of error for the EIM score is below an acceptable threshold, then the AIM is eligible for an EIM score for the provider group.

In some embodiments, the simulation determines the set of eligible EIMs using baseline data (e.g., data from 2020 in an example) for multiple large provider groups. In some embodiments, AIMs that are eligible for EIM scores tend to be measures that have large baseline racial/ethnic inequities and large denominators even when stratified by race/ethnicity across the provider groups.

### Section II. Detailed Description

In some embodiments, a set of measures eligible for an EIM score for a provider group is a set of the AIMs applied during the Measurement Period and that are calculable, or already calculated, at baseline. In some embodiments, each AIM satisfies the following two conditions to be eligible for an EIM score:
a. Condition 1: At least two racial/ethnic strata each have baseline denominator greater than 90.
b. Condition 2: The measure can have an average root mean squared error (RMSE) that is comparable or less than the RMSE tolerated by the AIMs at their MDRs after completing the simulations detailed below.

In some embodiments, the reliability calculation method used for the alternative AIM scores (e.g., a comparison of single proportions between providers at one point in time) are not used because the EIM score is based on within-provider improvements to inequities (e.g., the weighted average of differences between multiple proportions at two points in time). In some embodiments, the maximum threshold of magnitude of random error for EIM scores is comparable to the maximum threshold of magnitude of random error tolerated at the minimum denominator threshold for AIM scores because both EIMs and AIMs provide an output on a scale of 0-5. In some embodiments, if either of these two conditions is not satisfied for a provider group, then the AIM is not eligible for an EIM score for the provider group.

### 1. Condition 2—Simulation Set-Up

In some embodiments, Condition 2 is satisfied when the amount of measurement error associated with an EIM score for the provider group is comparable or less than the amount of error currently tolerated by the AIMs scores. In some embodiments, a Monte Carlo simulation examining a range of potential improvement patterns and Measurement Period Denominators is conducted because verification that Condition 2 is satisfied occurs before the Measurement Period. In some embodiments, the Monte Carlo simulation compares the true, known value of the EIM score for a specific assumed improvement pattern (g*) to a set of drawn EIM scores. In some embodiments, the set of drawn EIM scores are obtained by taking m draws of the Measurement Period data from the corresponding binomial probability distribution {g₁, g₂, . . . , gₘ}, given the assumed improvement pattern and Measurement Period denominators. In some embodiments, a specific set of Measurement Period Denominators is compared with an assumed improvement pattern using RMSE=\sqrt{MSE} when

$$MSE = \frac{1}{m} {\sum }_{i = 1}^{m} {( {g}_{i} - {g}^{*} )}^{2} = {( \overline{g} - {g}^{*} )}^{2} + \frac{1}{m} {\sum }_{i = 1}^{m} {( {g}_{i} - \overline{g} )}^{2} = {Bias}^{2} + Variance .$$

As a result, RMSE can be a measure of error that quantifies the estimator's bias and variability. In some embodiments, the simulation obtains an RMSE metric by averaging the RMSE across a range of improvement scenarios for a fixed set of Measurement Period denominators. In some embodiments, the RMSE metric from the simulation is used to determine if Condition 2 is satisfied.

In some embodiments, conducting the simulation for each provider group and calculating the average RMSE for each measure within each provider group is based on assumptions regarding the Measurement Period Denominators and Measurement because Measurement Period data are not available.

Period Stratified Performance rates: In some embodiments, assume that num_{RE}=4 and all racial/ethnic groups of Table 1 are included as part of the calculation. If num_{RE}< >4, these steps are completed in the same manner using the reduced set of racial/ethnic groups. In some embodiments, the following provides assumptions used to complete the simulation.
Assumption 1: In some embodiments, Measurement Period denominators vary by a constant, r, relative to Baseline denominators:

$${n}_{W , MP} = r ( {n}_{W , base} ) \\ {n}_{X , MP} = r ( {n}_{X , base} ) \\ {n}_{Y , MP} = r ( {n}_{Y , base} ) \\ {n}_{Z , MP} = r ( {n}_{Z , base} )$$

If r>1, then the denominators for all racial/ethnic groups in the Measurement Period can be greater than at baseline; if r<1, then the denominator value for all racial/ethnic groups in the Measurement Period can be less than at baseline. In general, r can range between 0.70 to 1.20, but other values for r are possible. In some embodiments, these analyses involved computing n_{t,2019}/n_{t,2018} for each t∈{Asian, Black, Hispanic, White}, measure, and provider group. In some embodiments, for each measure and each t∈{Asian, Black, Hispanic, White}, the minimum and maximum of n_{t,2019}/n_{t,2018} across all provider groups is calculated. In some embodiments, the average minimum of n_{t,2019}/n_{t,2018} across all measures was equal to 0.89 for Asian members, 0.80 for Black members, 0.90 for Hispanic members, 0.82 for White members. In some embodiments, the average maximum of n_{t,2019}/n_{t,2018} across all measures was equal to 1.15 for Asian members, 0.89 for Black members, 1.12 for Hispanic members, 1.11 for White members. In some embodiments, with the above minimums and maximums, the denominator change ratio r between the baseline and Measurement Period can be reasonably expected to be within the 0.70 to 1.20 range. In some embodiments, for purposes of the simulation, the range of r is therefore constant for all racial/ethnic groups to examine the most extreme case where all groups are experiencing declines in their denominators.
Assumption 2. In some embodiments, the simulation assumes that provider groups achieve equity improvements by improving Stratified Performance among the racial/ethnic groups for which baseline performance is worse by more than Stratified Performance among racial/ethnic groups for which baseline performance is better. In some embodiments, the simulation assumes that provider groups first improve performance for the racial/ethnic group receiving the lowest baseline performance until it matches the performance for the racial/ethnic group receiving the third-highest performance. In some embodiments, the simulation assumes that provider groups then improve performance for these two racial/ethnic groups until the performance for each matches the performance for the racial/ethnic group receiving the second-highest performance. In some embodiments, the simulation assumes that provider groups improve performance for these three racial/ethnic groups until the performance for each matches the performance of the racial/ethnic group receiving the highest performance. At this point in the simulation, it can be assumed zero inequities remain on this AIM.

In some embodiments, this ordered improvement path may not be the exact one a provider group may take because it is not possible to know exactly how any provider group may structure its improvement efforts in a new program. However, in some embodiments, the simulation is designed to the objective of the simulation was to examine variability across different levels of improvement. In some embodiments, an assumption is made about the pattern of improvement to run a simulation. In some embodiments, communication with provider groups incentivizes improving large baseline inequities or inequities with large Equity Weights.

In some embodiments, the simulation can assume different improvement path in which each inequity is reduced at a fixed percentage (e.g., 10%, 20%, . . . ) is also considered. In some embodiments, compared to the selected improvement path, the RMSEs resulting from this alternative improvement path were similar.

In some embodiments, certain improvement steps are provided for a measure in which higher values of p indicate better performance.
1. In some embodiments, the set of Baseline Stratified Performance rates, {p_{W,base}, p_{X,base}, p_{Y,base}, p_{Z,base}}, are sorted such that p_{1,base} is the racial/ethnic group receiving the highest performance, p_{2,base} is the racial/ethnic group receiving the second-highest performance, p_{3,base} is the racial/ethnic group receiving the third-highest performance, and p_{4,base} is the racial/ethnic group receiving the lowest performance.
2. In some embodiments, the difference between the groups receiving the highest and lowest performances, p_{1,base}−p_{4,base} is rounded to the nearest thousandth. In some embodiments, this difference is divided in proportions by 0.001, and then 1 is added to this value. In some embodiments, the number obtained is equal to the number of rows in a matrix representing the potential combinations of Measurement Period Stratified Performance values given the assumed improvement pattern. In some embodiments, the number of columns is equal to num_{RE}.
3. In some embodiments, for the column corresponding to the racial/ethnic group receiving the highest performance, all entries of the matrix equal to p_{1,base}. In some embodiments, for this group, the Measurement Period Stratified Performance is assumed to be equal to the Baseline Stratified Performance in all simulation settings.
4. In some embodiments, for the racial/ethnic group receiving the lowest performance, its corresponding column in the matrix representing potential Measurement Period Stratified Performance should be equal to the sequence from p_{4,base} to p_{1,base}, increasing by 0.001 in each row.
5. In some embodiments, for the column corresponding to the group receiving the third highest performance, all rows for which the group receiving the lowest Stratified Performance has performance less than or equal to p_{3,base} should be equal to p_{3,base}. In some embodiments, once the group receiving the lowest Stratified Performance catches up to the group receiving the third highest performance, both groups should move together to improve their performance until they reach p_{1,base}.
6. In some embodiments, a similar pattern is assumed for the column corresponding to the racial/ethnic group receiving the second highest performance: once the groups receiving the lowest and third highest performance reach p_{2,base} performance. In some embodiments, the Measurement Period Stratified Performance for the groups receiving the 2^{nd}, 3^{rd} and 4^{th} highest performances are assumed to improve up until they reach that of the group receiving the highest performance.

In some embodiments, the table below illustrates a matrix having the full set of potential Measurement Period Stratified Performance (MPSP in the tables) values, given this assumed pattern of improvement. In some embodiments, in the first row of this matrix, the EIM score is 0 because all Measurement Period Stratified Performance values are equal to their baseline values (and so inequities stay equal as well). In the last row of this matrix, the EIM score is equal to 5 because there are no inequities remaining.

TABLE 6
Pattern of Improvement Matrix

| | Potential MPSP for | Potential MPSP for | Potential MPSP for | Potential MPSP for |
|---|---|---|---|---|
| | Group Receiving | Group Receiving | Group Receiving | Group Receiving |
| | Highest | 2^{nd} Highest | 3^{rd} Highest | Lowest |
| | Performance | Performance | Performance | Performance |
| Number | p_{1, base} | p_{2, base} | p_{3, base} | p_{4, base} |
| of rows | p_{1, base} | p_{2, base} | p_{3, base} | p_{4, base} + 0.001 |
| is equal | p_{1, base} | p_{2, base} | p_{3, base} | p_{4, base} + 0.002 |
| to | . . . | . . . | . . . | . . . |
| ((p_{1, base} − | p_{1, base} | p_{2, base} | p_{3, base} | p_{3, base} |
| p_{4, base})/ | p_{1, base} | p_{2, base} | p_{3, base} + 0.001 | p_{3, base} + 0.001 |
| 0.001) + 1 | p_{1, base} | p_{2, base} | p_{3, base} + 0.002 | p_{3, base} + 0.002 |
| | . . . | . . . | . . . | . . . |
| | p_{1, base} | p_{2, base} | p_{2, base} | p_{2, base} |
| | p_{1, base} | p_{2, base} + 0.001 | p_{2, base} + 0.001 | p_{2, base} + 0.001 |
| | p_{1, base} | p_{2, base} + 0.002 | p_{2, base} + 0.002 | p_{2, base} + 0.002 |
| | . . . | . . . | . . . | . . . |
| | p_{1, base} | p_{1, base} | p_{1, base} | p_{1, base} |

Number of columns is equal to num_{RE}

Three non-limiting examples below illustrate how various embodiments of the simulation is configured for specific values of r and the assumed improvement pattern.

### Example 1

In this example, r=1.0 and the data available at baseline for the provider group are as illustrated in Table 7.

TABLE 7
Example 1 Baseline Data

| | Denominator | | Stratified Performance | | | | Category Inequity | | |
|---|---|---|---|---|---|---|---|---|---|
| | | Measurement | | Measurement | | | | Measurement | Equity |
| | Baseline | Period | Baseline | Period | MDR | MPR | Baseline | Period | Weights |
| Asian | 180 | | 0.650 | | 144 | 0.565 | 0.100 | | 0.657 |
| Black | 150 | | 0.720 | | 120 | 0.632 | 0.030 | | 0.343 |
| Hispanic | 85 | | 0.680 | | NA | NA | NA | | NA |
| White | 2000 | | 0.750 | | 1600 | 0.727 | NA | | NA |

| | |
|---|---|
| Baseline Weighted Average Inequity | 0.076 |
| Weighted Average Inequity | |

In the example illustrated by Table 7, White is the reference group in this example because its baseline denominator is the largest. In addition, Hispanic members are excluded from calculations because n_{Hispanic,base}<90.
1. In some embodiments, in the first simulation step, it is determined that the Measurement Period Denominators are each greater than the MDR because r=1.0,

$${n}_{White , MP} = {n}_{White , base} , {n}_{Black , MP} = {n}_{Black , base} = {n}_{Asian , MP} = {n}_{Asian , base} .$$

2. In some embodiments, in the next simulation step, the Measurement Period Stratified Performance rates are each greater than the MPRs across all combinations of the matrix below based on the assumed improvement pattern. Based on the data of the example, the White racial/ethnic group receives the highest performance and the Asian racial/ethnic group receives the lowest performance.
3. In some embodiments, the EIM score is calculated for each row of the potential Measurement Period Stratified Performance rates and other r values based on the simulation. Table 8 illustrates an example of results of embodiments of the simulation. In Table 8, the first row is the first iteration, and each successive row is a successive iteration.

TABLE 8
Example 1 Simulation Data

| | Potential MPSP for | Potential MPSP for | Potential MPSP for |
|---|---|---|---|
| | Group Receiving | Group Receiving | Group Receiving |
| | Highest | 2^{nd} Highest | Lowest |
| | Performance White | Performance Black | Performance Asian |
| Number | 0.750 | 0.720 | 0.650 |
| of rows | 0.750 | 0.720 | 0.650 + 0.001 |
| is equal | 0.750 | 0.720 | 0.650 + 0.002 |
| to 101 | . . . | . . . | . . . |
| | 0.750 | 0.720 | 0.720 |
| | 0.750 | 0.720 + 0.001 | 0.720 + 0.001 |
| | 0.750 | 0.720 + 0.002 | 0.720 + 0.002 |
| | . . . | . . . | . . . |
| | 0.750 | 0.750 | 0.750 |

Number of columns is equal to num_{RE} = 3

### Example 2

In this example, r=1.2 and the data available at baseline for the provider group are as illustrated in Table 9.

TABLE 9
Example 2 Baseline Data

| | Denominator | | Stratified Performance | | | | Category Inequity | | |
|---|---|---|---|---|---|---|---|---|---|
| | | Measurement | | Measurement | | | | Measurement | Equity |
| | Baseline | Period | Baseline | Period | MDR | MPR | Baseline | Period | Weights |
| Asian | 180 | | 0.850 | | 144 | 0.784 | 0.100 | | 0.418 |
| Black | 150 | | 0.790 | | 120 | 0.707 | 0.040 | | 0.235 |
| Hispanic | 250 | | 0.800 | | 200 | 0.737 | 0.050 | | 0.347 |
| White | 2000 | | 0.750 | | 1600 | 0.726 | NA | | NA |

| | |
|---|---|
| Baseline Weighted Average Inequity | 0.069 |
| Weighted Average Inequity | |

In the example illustrated by Table 9, White is the reference group because its baseline denominator is the largest of the four racial/ethnic groups. In this example, all four racial/ethnic groups are included in calculations because the baseline denominators for each are greater than 90.
1. In some embodiments, in the first simulation step, it is determined that the Measurement Period Denominators are each greater than the MDR because r=1.2, n_{Asian,MP}=1.2n_{Asian, base}, n_{Black,MP}=1.2n_{Black,base}, n_{Hispanic,MP}=1.2n_{Hispanic,base}, n_{White,MP}=1.2n_{White,base}.
2. In some embodiments, the Measurement Period Stratified Performance rates are each larger than the MPRs across all combinations of the matrix below based on the assumed improvement pattern. In some embodiments, as illustrated by Table 10 below, the Asian racial/ethnic group initially receives a highest performance metric and the White racial/ethnic group receives the lowest performance.
3. In some embodiments, the EIM score is calculated for each row of the potential Measurement Period Stratified Performance rates and other r values based on the simulation. Table 10 illustrates an example of results of embodiments of the simulation. In Table 10, the first row is the first iteration, and each successive row is a successive iteration.

TABLE 10
Example 2 Simulation Data

| | Potential MPSP for | Potential MPSP for | Potential MPSP for | Potential MPSP for |
|---|---|---|---|---|
| | Group Receiving | Group Receiving | Group Receiving | Group Receiving |
| | Highest | 2^{nd} Highest | 3^{rd} Highest | Lowest |
| | Performance Asian | Performance Hispanic | Performance Black | Performance White |
| Number | 0.850 | 0.800 | 0.790 | 0.750 |
| of rows | 0.850 | 0.800 | 0.790 | 0.750 + 0.001 |
| is equal | 0.850 | 0.800 | 0.790 | 0.750 + 0.002 |
| to 101 | . . . | . . . | . . . | . . . |
| | 0.850 | 0.800 | 0.790 | 0.790 |
| | 0.850 | 0.800 | 0.790 + 0.001 | 0.790 + 0.001 |
| | 0.850 | 0.800 | 0.790 + 0.002 | 0.790 + 0.002 |
| | . . . | . . . | . . . | . . . |
| | 0.850 | 0.800 | 0.800 | 0.800 |
| | 0.850 | 0.800 + 0.001 | 0.800 + 0.001 | 0.800 + 0.001 |
| | 0.850 | 0.800 + 0.002 | 0.800 + 0.002 | 0.800 + 0.002 |
| | . . . | . . . | . . . | . . . |
| | 0.850 | 0.850 | 0.850 | 0.850 |

Number of columns is equal to num_{RE} = 4

### Example 3

In this example, r=0.8 and the data available at baseline for the provider group are as illustrated in Table 11.

TABLE 11
Example 3 Baseline Data

| | Denominator | | Stratified Performance | | | | Category Inequity | | |
|---|---|---|---|---|---|---|---|---|---|
| | | Measurement | | Measurement | | | | Measurement | Equity |
| | Baseline | Period | Baseline | Period | MDR | MPR | Baseline | Period | Weights |
| Asian | 180 | | 0.850 | | 144 | 0.784 | 0.100 | | 0.418 |
| Black | 150 | | 0.790 | | 120 | 0.707 | 0.040 | | 0.235 |
| Hispanic | 250 | | 0.700 | | 200 | 0.628 | 0.050 | | 0.347 |
| White | 2000 | | 0.750 | | 1600 | 0.726 | NA | | NA |

| | |
|---|---|
| Baseline Weighted Average Inequity | 0.069 |
| Weighted Average Inequity | |

In the example illustrated by Table 11, White is the reference group because its baseline denominator is the largest. In the example illustrated by Table 11, all four racial/ethnic groups are included in calculations because the baseline denominators for each are greater than 90.

In some embodiments, the Measurement Period Denominators are each equal to the MDR because r=0.8, n_{Asian,MP}=0.8n_{Asian,base}, n_{Black,MP}=0.8n_{Black,base}, n_{Hispanic,MP}=0.8n_{Hispanic,base} n_{White,MP}=0.8n_{White,base}. In some embodiments, this calculation can be performed because none of the Measurement Period Denominators are less than their corresponding MDRs.

In some embodiments, the Measurement Period Stratified Performance rates are each larger than the MPRs across all combinations of the matrix below based on the assumed improvement pattern. In some embodiments, as illustrated by Table 12 below, the Asian racial/ethnic group receives the highest performance and the Hispanic racial/ethnic group receives the lowest performance in a first iteration of an embodiment of the simulation.

In some embodiments, the EIM score is calculated for each row of the potential Measurement Period Stratified Performance rates and other r values (e.g., between 0.70 and 1.20) based on the simulation.

TABLE 12
Example 3 Simulation Data

| | Potential MPSP for | Potential MPSP for | Potential MPSP for | Potential MPSP for |
|---|---|---|---|---|
| | Group Receiving | Group Receiving | Group Receiving | Group Receiving |
| | Highest | 2^{nd} Highest | 3^{rd} Highest | Lowest |
| | Performance Asian | Performance Black | Performance White | Performance Hispanic |
| Number | 0.850 | 0.790 | 0.750 | 0.700 |
| of rows | 0.850 | 0.790 | 0.750 | 0.700 + 0.001 |
| is equal | 0.850 | 0.790 | 0.750 | 0.700 + 0.002 |
| to 151 | . . . | . . . | . . . | . . . |
| | 0.850 | 0.790 | 0.750 | 0.750 |
| | 0.850 | 0.790 | 0.750 + 0.001 | 0.750 + 0.001 |
| | 0.850 | 0.790 | 0.750 + 0.002 | 0.750 + 0.002 |
| | . . . | . . . | . . . | . . . |
| | 0.850 | 0.790 | 0.790 | 0.790 |
| | 0.850 | 0.790 + 0.001 | 0.790 + 0.001 | 0.790 + 0.001 |
| | 0.850 | 0.790 + 0.002 | 0.790 + 0.002 | 0.790 + 0.002 |
| | . . . | . . . | . . . | . . . |
| | 0.850 | 0.850 | 0.850 | 0.850 |

Number of columns is equal to num_{RE} = 4

### 2. Condition 2—Implementing the Simulation

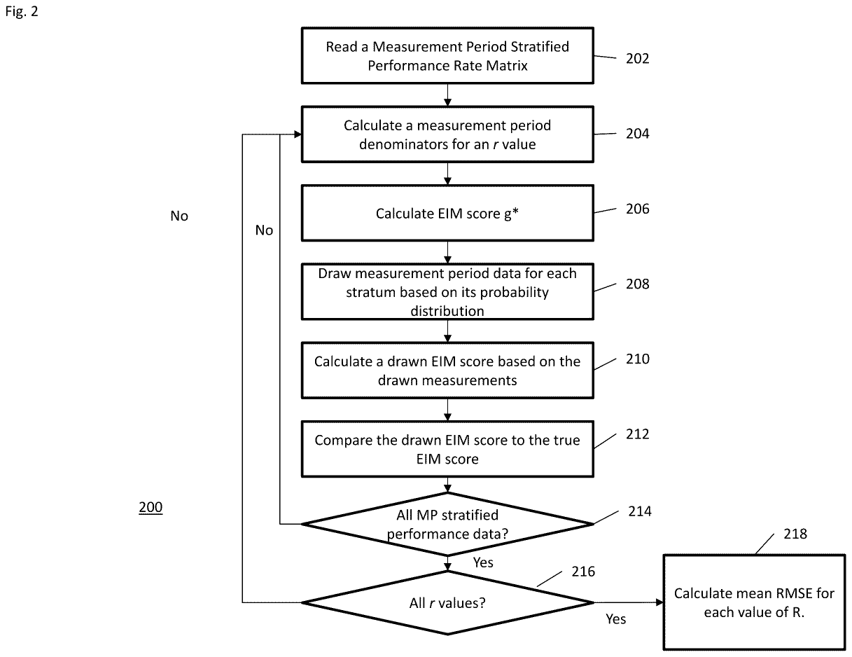

FIG. 2 is a flowchart 200 illustrating an example of a method of running a simulation to determine whether a condition is satisfied for a provider group according to embodiments of the present disclosure. In some embodiments, the method can be performed by a server or other computer. In some embodiments, a Monte Carlo simulation determines whether Condition 2 is satisfied for the provider group for each AIM that satisfies Condition 1.

In some embodiments, the simulation calculates the baseline calculations (e.g., Minimum Denominator Requirement (MDR), Minimum Performance Required (MPR), Baseline Category Inequity, Baseline Denominator Weights, Baseline Category Inequity Weights, Equity Weights, and/or Baseline Weighted Average Inequity) described above.

In some embodiments, each row of a matrix of potential Measurement Period Stratified Performance rates is evaluated for each value of r∈{0.7, 0.8, 0.9, 1.0, 1.1, 1.2}.

In some embodiments, the evaluation of each row of the matrix for one of the potential values of r includes the following steps. In some embodiments, the evaluation includes calculating the Measurement Period Denominators for this value of r 202. In some embodiments, the evaluation calculates the true EIM score, g*, using the Baseline and Measurement Period Denominators and Stratified Performance 206.

In some embodiments, the evaluation draws Measurement Period Denominators and Stratified Performance for each racial/ethnic stratum based on its probability distribution 208. In some embodiments, the number of replications is set to m=20,000. In some embodiments, the evaluation performs 20,000 draws of Binomial (n_{w,MP}, p_{w,MP}), 20,000 draws of Binomial (n_{x,MP}, p_{x,MP}, 20,000 draws of Binomial (n_{y,MP}, p_{y,MP}), and 20,0000 draws of Binomial (n_{z,MP}, p_{z,MP}).

In some embodiments, for each set of draws, in combination with the observed baseline denominators and Stratified Performance Rates, the evaluation calculates the drawn EIM score gᵢ 210. In some embodiments, the evaluation then compares the m=20,000 draws of the EIM score, {g₁, g₂, . . . , g_{20,000}}, to the true EIM score g* using RMSE=\sqrt{MSE}, where

$$MSE = \frac{1}{m} {\sum }_{i = 1}^{m} {( {g}_{i} - {g}^{*} )}^{2} 212 .$$

In some embodiments, the evaluation iterates for each row of the matrix 214. In some embodiments, the evaluation iterates to perform the evaluation for all potential r values 216. In some embodiments, the evaluation performs the calculation for all r values with a one-shot calculation without iteration.

In some embodiments, the evaluation calculates the mean RMSE for each fixed value of r 218. In some embodiments, the RMSE calculation excludes any combinations that lead to above a 95% reduction in PRW because the objective is to learn about realistic improvements in equity that would take place in the initial years of the program. In some embodiments, for a specific value of r, this is equal to the mean of the RMSEs from each of the combinations of Measurement Period Stratified Performance.

### 3. Using Simulation Results

In some embodiments, if the average RMSE at any value of r≤1 is comparable in magnitude to the RMSE tolerated by the AIMs at their MDRs, then the measure is eligible for equity calculations for the provider group because the degree of error associated to this EIM is similar in magnitude to the degree of error tolerated in AIMs scores. In some embodiments, among measures that are eligible, the MDRs for that measure, r_{MDR}(n_{t,base}), are calculated by multiplying baseline denominators by the smallest value of r=0.7, 0.8, 0.9, 1.0 associated to the RMSE tolerated by the AIMs at their MDRs.

In some embodiments, the RMSE tolerated by the AIMs at their MDRs are calculated based on a separate Monte Carlo simulation examining the degree of uncertainty currently accepted in the AIM scores. In some embodiments, each measure that receives an AIM score has a Minimum Denominator Required, Minimum Threshold and Upper Threshold. In some embodiments, for each Provider and each of the measures that receives an AIM score, the Actual Provider Performance Level (p_{AIM}) and the AIM Minimum Denominator Required are used for 1000 draws from the binomial distribution and obtain a set of 1000 drawn performance rates, p_{AIM,i} for i=1, . . . , 1000. In some embodiments, these drawn performance rates are then converted to a score between 0 and 5 (similar to the EIM score formula) by letting

$${PRW}_{AIM , i} = \frac{{p}_{AIM , i} - Minimum Thresho1d}{Upper Threshold - Minimum Threshold}$$

and using the formula: Score=6.667(PRW_{AIM,i}) when PRW_{AIM,i}<0.75 and Score=5 when PRW_{AIM,i}≥0.75. In some embodiments, if p_{AIM,i} is less than the Minimum Threshold, then the Score is replaced with 0; if p_{AIM,i} is greater than the Upper Threshold then the Score is replaced with 5. In some embodiments, the 1000 drawn Scores are compared to the true Score (derived using the observed p_{AIM} and the Score formula) using the RMSE. In some embodiments, the mean RMSE was then calculated across all groups and all measures to inform the mean RMSE threshold used to determine a measure's eligibility for an EIM score.

### Determining the Curve Shape for the EIM Score

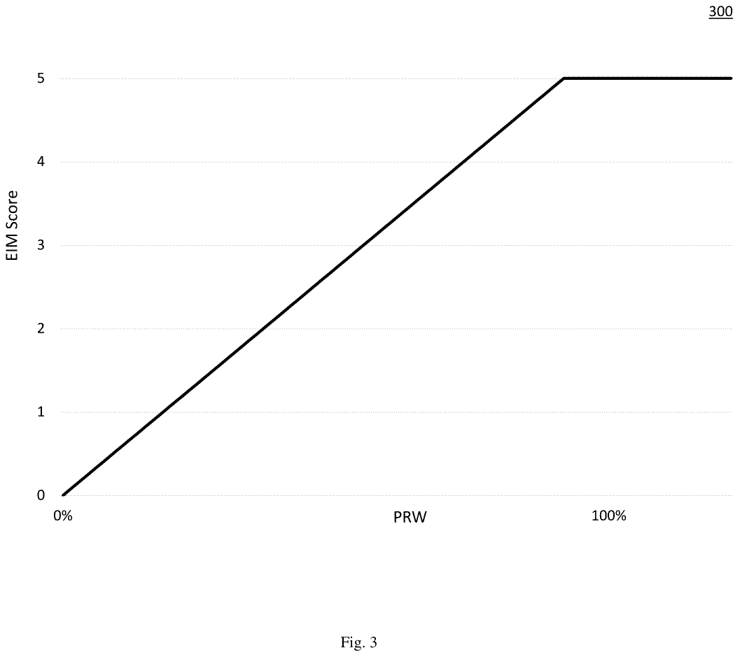

FIG. 3 is a graph 300 of the EIM score plotted against the PRW in accordance with embodiments of the present disclosure. In some embodiments, transforming the percent reduction in weighted baseline inequity (PRW) to an EIM Score is based on the following steps. In some embodiments, the conversion from PRW to EIM score can be set to be generous to provider groups because race/ethnicity data are fully imputed and therefore may contain measurement errors. In some embodiments, the EIM Score is 6.667 (PRW) when PRW is less than 75% and the EIM Score is 5 when PRW is greater than 75%. In some embodiments, rather than require the PRW to be 100% to earn an EIM score equal to 5 (e.g., the maximum EIM score), the PRW needs to be 75% or more. In some embodiments, setting the PRW to be 75% for a maximum score makes it easier to earn the maximum payout to account for the use of fully imputed race/ethnicity data. The Plan considers increasing this threshold as more self-reported data become available.

In some embodiments, the decision to transform any PRW score greater than or equal to 75% (or equivalently 0.75) to an EIM Score of 5 is based on an analysis that examined book-of-business data for a subset of measures with some self-reported race/ethnicity data available. In some embodiments, for this group of measures and using only members with self-reported race/ethnicity, a scenario is modeled in which the Plan was using fully imputed race/ethnicity data to determine baseline weighted inequity values and to track improvement, while the provider organization (under the assumption that the provider organization will have 100% self-reported race/ethnicity data during the Measurement Period) is using only true self-reported race/ethnicity data to guide and track internal improvement efforts. In some embodiments, the Plan's calculations may not match the true PRW values.

In some embodiments, for each measure, the analyses compared how simulated improvements to the true PRW calculated using self-reported race/ethnicity translated to changes to the PRW that is calculated using imputed race/ethnicity. In some embodiments, the PRW is calculated using only self-reported race/ethnicity data approached 100%, the PRW calculated using only imputed data approached 75% across most measures examined.

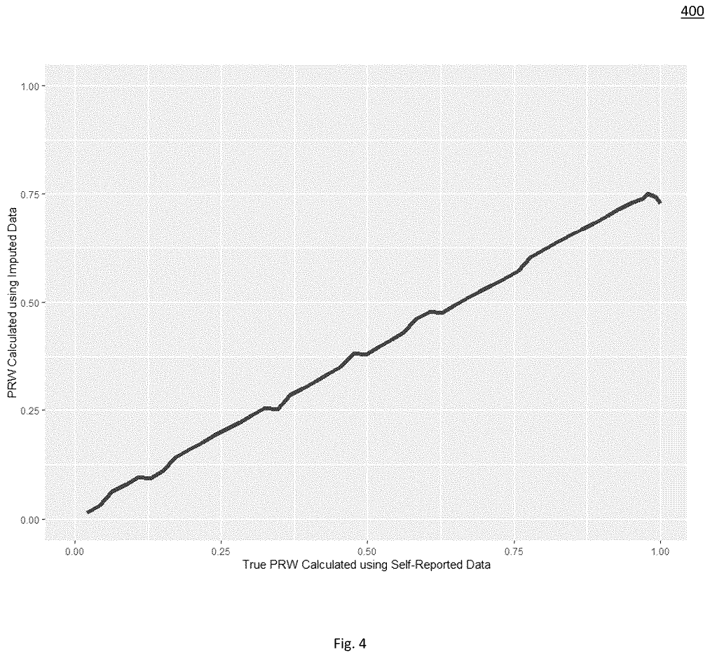

FIG. 4 is a graph 400 illustrating PRW calculating using imputed data plotted against PRW plotted using self-reported data. The graph 400 is an illustrative example of results for an analysis for a group of 22,184 members for the cervical cancer screening measure. The group of this illustrative example reported race and ethnicity. The graph demonstrates that, in some embodiments, if the provider organization has internal access to self-reported data and is using the EIM score to guide their improvement, as they come close to achieving a PRW of 100%, the Plan, which is using imputed data, estimates the PRW to be closer to about 75%. The curve connecting the PRW to the EIM Score has been transformed to account for this underestimation because the Plan is limited to using imputed race/ethnicity due to high levels of missingness for self-reported race/ethnicity data.

In some embodiments, it can be reasonable to assume that if the 100% self-reported race/ethnicity data were available and used to calculate the PRW instead of imputed data for the graph 300 of FIG. 3, the segmented lines would be closer to a straight line between 0 and 5. However, because imputed race/ethnicity is being used, the line currently displayed FIG. 3 reaches an EIM Score of 5 at PRW=75% and has a steeper slope between a PRW of 0 and 75% than it would if it were a straight line between 0 and 100%.

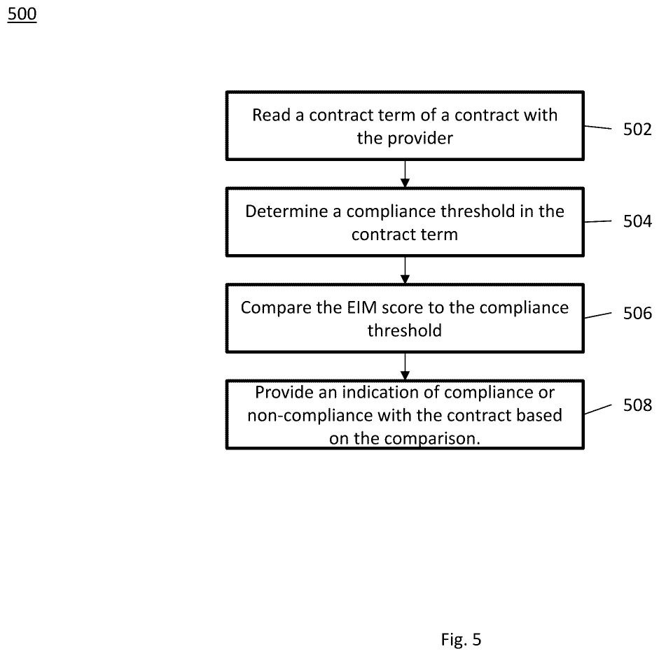

FIG. 5 is a flowchart 500 illustrating a method of using the EIM score to comply with a contract according to embodiments of the present disclosure. In some embodiments, the method can be employed by a contract compliance server that is in communication with the provider via a network such as the Internet. The contract compliance server can read a contract term of a contract with the provider 502. The contract compliance server can determine a compliance threshold in the contract term 504. The contract compliance server can compare the EIM score to the compliance threshold 506. The contract compliance server can provide an indication of compliance or non-compliance with the contract based on the comparison 508. In some embodiments, the contract compliance server can also calculate the EIM score (e.g. according to the method illustrated by FIG. 1). In some embodiments, the contract compliance server can receive the EIM score from another source, such as another server.

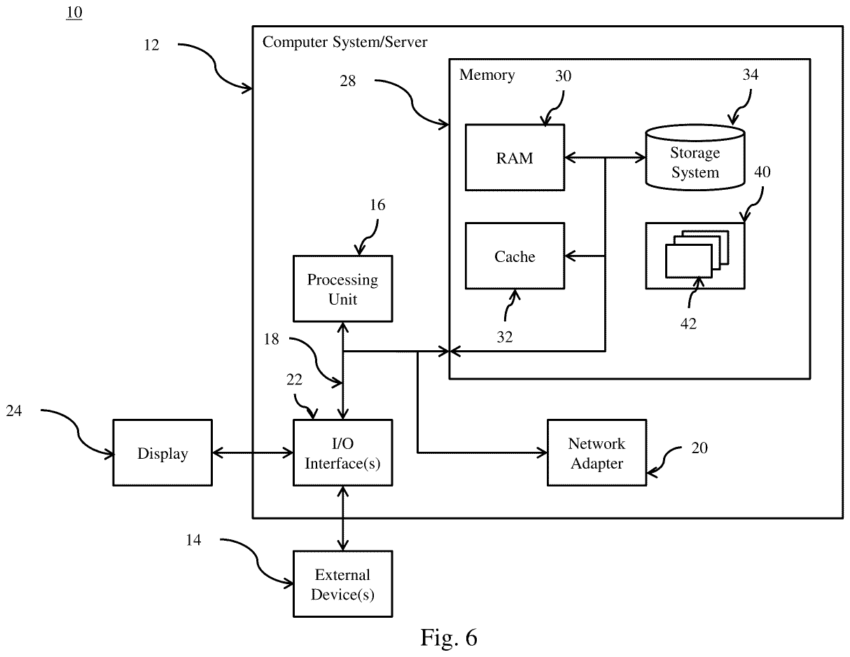

FIG. 6 is a computing node 10 according to embodiments of the present disclosure. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 6, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method of assigning an Equity Incentive Measure (EIM) score to a provider, the method comprising:
reading a first size and a first performance score associated with each of a plurality of groups in a baseline time period;
reading a second size and a second performance score associated with each of the plurality of groups in a measurement time period;
filtering the plurality of groups to those whose first and second size exceeds a predetermined size threshold;
filtering the plurality of groups to those whose first and second performance score exceeds a predetermined performance threshold;
selecting one of the plurality of groups as a reference group;
determining a first average inequity for each of the plurality of groups relative to the reference group based on their respective first size and first performance score;
determining a second average inequity for each of the plurality of groups relative to the reference group based on their respective second size and second performance score; and
determining an EIM score based on a percentage change in inequity between the first and the second average inequities.

2. The method of claim 1, wherein determining the first average inequity for each of the plurality of groups comprises:
determining an equity weight for each of the plurality of groups relative to the reference group based on their respective first size and first performance score;
determining a weight for each of the plurality of groups relative to the reference group, based on their respective first size; and
determining a baseline inequity for each of the plurality of groups relative to the reference group based on their respective first size and first performance score.

3. The method of claim 1, wherein determining the second average inequity for each of the plurality of groups comprises:
determining an equity weight for each of the plurality of groups relative to the reference group based on their respective second size and second performance score;
determining a weight for each of the plurality of groups relative to the reference group, based on their respective second size; and
determining a baseline inequity for each of the plurality of groups relative to the reference group based on their respective second size and second performance score.

4. The method of claim 1, wherein determining the EIM score is a constant if the percentage change in inequity is at or above a threshold.

5. The method of claim 4, wherein the EIM score is a product of the percentage change in inequality between the first and second average inequities and a linear constant if the percentage change in inequity is below the threshold.

6. The method of claim 1, wherein the first performance score and second performance score are each a quality improvement metric.

7. The method of claim 1, further comprising:
reading a contract term of a contract with the provider;
determining a compliance threshold in the contract term;
comparing the EIM score to the compliance threshold; and
providing an indication of compliance or non-compliance with the contract based on the comparing.

8. A non-transitory computer readable storage medium storing executable instructions that, when executed by one or more processors, cause the one or more processors to perform a method of assigning an Equity Incentive Measure (EIM) score to a provider, the method comprising:
reading a first size and a first performance score associated with each of a plurality of groups in a baseline time period;
reading a second size and a second performance score associated with each of the plurality of groups in a measurement time period;
filtering the plurality of groups to those whose first and second size exceeds a predetermined size threshold;
filtering the plurality of groups to those whose first and second performance score exceeds a predetermined performance threshold;
selecting one of the plurality of groups as a reference group;
determining a first average inequity for each of the plurality of groups relative to the reference group based on their respective first size and first performance score;
determining a second average inequity for each of the plurality of groups relative to the reference group based on their respective second size and second performance score; and
determining an EIM score based on a percentage change in inequity between the first and the second average inequities.

9. (canceled)

10. The non-transitory computer readable storage medium of claim 8, wherein determining the first average inequity for each of the plurality of groups comprises:
determining an equity weight for each of the plurality of groups relative to the reference group based on their respective first size and first performance score;
determining a weight for each of the plurality of groups relative to the reference group, based on their respective first size; and
determining a baseline inequity for each of the plurality of groups relative to the reference group based on their respective first size and first performance score.

11. The non-transitory computer readable storage medium of claim 8, wherein determining the second average inequity for each of the plurality of groups comprises:
determining an equity weight for each of the plurality of groups relative to the reference group based on their respective second size and second performance score;
determining a weight for each of the plurality of groups relative to the reference group, based on their respective second size; and
determining a baseline inequity for each of the plurality of groups relative to the reference group based on their respective second size and second performance score.

12. The non-transitory computer readable storage medium of claim 8, wherein determining the EIM score is a constant if the percentage change in inequity is at or above a threshold.

13. The non-transitory computer readable storage medium of claim 12, wherein the EIM score is a product of the percentage change in inequality between the first and second average inequities and a linear constant if the percentage change in inequity is below the threshold.

14. The non-transitory computer readable storage medium of claim 8, wherein the first performance score and second performance score are each a quality improvement metric.

15. The non-transitory computer readable storage medium of claim 8, wherein the method performed by the processor further comprises:
reading a contract term of a contract with the provider;
determining a compliance threshold in the contract term;
comparing the EIM score to the compliance threshold; and
providing an indication of compliance or non-compliance with the contract based on the comparing.

16. A system comprising:
a computing node comprising a computer readable storage medium having program instructions embodied therewith, the program instructions executable by a processor of the computing node to cause the processor to perform a method comprising:
reading a first size and a first performance score associated with each of a plurality of groups in a baseline time period;
reading a second size and a second performance score associated with each of the plurality of groups in a measurement time period;
filtering the plurality of groups to those whose first and second size exceeds a predetermined size threshold;
filtering the plurality of groups to those whose first and second performance score exceeds a predetermined performance threshold;
selecting one of the plurality of groups as a reference group;
determining a first average inequity for each of the plurality of groups relative to the reference group based on their respective first size and first performance score;
determining a second average inequity for each of the plurality of groups relative to the reference group based on their respective second size and second performance score; and
determining an EIM score based on a percentage change in inequity between the first and the second average inequities.

17. The system of claim 16, wherein determining the first average inequity for each of the plurality of groups comprises:
determining an equity weight for each of the plurality of groups relative to the reference group based on their respective first size and first performance score;
determining a weight for each of the plurality of groups relative to the reference group, based on their respective first size; and
determining a baseline inequity for each of the plurality of groups relative to the reference group based on their respective first size and first performance score.

18. The system of claim 16, wherein determining the second average inequity for each of the plurality of groups comprises:
determining an equity weight for each of the plurality of groups relative to the reference group based on their respective second size and second performance score;
determining a weight for each of the plurality of groups relative to the reference group, based on their respective second size; and
determining a baseline inequity for each of the plurality of groups relative to the reference group based on their respective second size and second performance score.

19. The system of claim 16, wherein determining the EIM score is a constant if the percentage change in inequity is at or above a threshold.

20. The system of claim 19, wherein the EIM score is a product of the percentage change in inequality between the first and second average inequities and a linear constant if the percentage change in inequity is below the threshold.

21. The system of claim 16, wherein the first performance score and second performance score are each a quality improvement metric.
